(19) United States
(12) Patent Application Publication
(10) Pub. No.: US 2007/0072195 A1
(43) Pub. Date: Mar. 29, 2007

(54) Bio-commemorative article incorporating amplified genetic material

(76) Inventors: Constantine Haralambopoulos, Rochester, NY (US); Barbara McDowell, Jeffersonton, VA (US); Marc A. Rossi, West Palm Beach, FL (US)

Correspondence Address: ROSSI, KIMMS & McDOWELL LLP., P.O. BOX 826, ASHBURN, VA, 20146-0826, US

(21) Appl. No.: 11/238,627

(22) Filed: Sep. 29, 2005

(51) Int. Cl. C12Q 1/68; G09F 19/00; C12P 19/34

(52) U.S. Cl. 435/6; 435/91.2; 428/7

(57) ABSTRACT

A combination of amplified genetic material from an individual or other organism with an inanimate article of manufacture to produce a bio-commemorative article is disclosed. The amplified genetic material is preferably produced from a polymerase chain reaction process. The amplified genetic material is coated onto the inanimate article of manufacture, incorporated within a structure of the inanimate article of manufacture or contained within a chamber provided in the inanimate article of manufacture. Optionally, the bio-commemorative article is packaged with documentation that establishes authenticity of the genetic material.

### BACKGROUND OF THE INVENTION

The invention relates generally to a combination of amplified genetic material from an individual or other organism with an inanimate article of manufacture to produce a bio-commemorative article, and, more particularly, to a combination of amplified genetic material from a specific individual or organism with an inanimate article of manufacture and documentation that establishes authenticity of the genetic material. The bio-commemorative articles are primarily designed for entertainment, recreation and amusement.

Collecting objects owned by or associated with famous individuals is popular. Furnishings from the estates of celebrities, for example, frequently fetch large sums far beyond their intrinsic value as objects. Items of clothing or jewelry actually worn by an individual are likewise highly valued by collectors and fans. Especially desirable are items, such as hair, that actually belonged to the individual of interest. For example, hair clippings from Elvis Presley recently sold for an amount in excess of $100,000, and another hair sample and a tooth from Mr. Presley fetched a similar amount. As can well be imagined, there is a very limited supply of such items, and thus their cost is typically beyond the reach of much of the population, which nonetheless desires to own an item that is intimately connected with the famous individual.

In fact, there is believed to be a ready market for items not only directly associated with a famous individual, such has a lock of hair, but even items associated in a subsidiary fashion, such as a flower from a persons grave or grass from the home of the individual. Of course, such items are of limited supply when weighed against a potentially huge market of worldwide admirers. Accordingly, it would be desirable to provide articles of manufacture and a method of producing the same that could be directly or indirectly associated with the famous person or their estate.

Further, the desire to collect commemorative objects is not limited to those related to famous individuals. For example, some people might wish to collect objects related to their pets, rare species of plants or even extinct creatures such as dinosaurs. Accordingly, it would further be desirable to provide an article of manufacture and method of producing the same that could be directly or indirectly associated with any type of organism whether or not presently alive or extinct.

### SUMMARY OF THE INVENTION

Recently there have been advances in the methods for obtaining genetic material from hair and other samples where the amount of nuclear DNA is small. Techniques now exist for amplifying even tiny amounts of genetic material, such as sweat or skin cells from an article of clothing worn by the individual. The present invention utilizes such techniques in order to replicate or amplify genetic material associated with an individual or other organism and incorporates the amplified genetic material in an article of manufacture.

In a preferred embodiment, there is provided a combination, comprising an article of manufacture and amplified genetic material from an organism, wherein the combination is a bio-commemorative article designed for entertainment, recreation and amusement. Preferably the genetic material is DNA. The combination may be packaged with accompanying documentation establishing the origin of the genetic material. This documentation may comprise a chain of title for the material from which the genetic material was extracted, or it may comprise evidence of a comparison to DNA from a close relative of the individual.

The bio-commemorative article may comprise, for example, an charm or other form of jewelry, a box, a vase or similar articles. Further, since the DNA in the article of manufacture may be mixed with another material—for example a solid, liquid or gel—, which may be colored, colorless, opaque or transparent, the DNA can be incorporated into or onto articles by molding, casting, coating, printing, painting or physical attachment.

In a preferred embodiment, the DNA of a famous person is utilized and the article of manufacture may include the name and/or picture of the person. However, DNA from any type of organism (even extinct organisms) can be incorporated into a bio-commemorative article. It will be understood for the purposes of this discussion, that the term “organism” includes any living thing from lower order organism such as bacteria to higher order organisms such as humans.

The sample DNA from which the amplified DNA is produced may be obtained from various sources. In the case of an individual, these sources include, but are not limited to, hair from the individual, a tooth from the individual, or an article of clothing that was worn by the individual. In other cases, for example plants, the DNA may be collected from pollen either directly from the plant or from a location adjacent thereto. Still further, DNA may be extracted from insects or other organisms trapped in amber or from remains of extinct species. The possibilities are endless and will become apparent to those skilled in the art from the following detailed description of the preferred embodiments of the invention.

### BRIEF DESCRIPTION OF THE DRAWINGS

The foregoing advantages and features of the invention will become apparent upon reference to the following detailed description and the accompanying drawings, of which:

FIG. 1 is an example of a bio-commemorative article according to a preferred embodiment of the invention; and

FIG. 2 is an example of a baseball card including amplified DNA in a printed portion of the baseball card.

### DETAILED DESCRIPTION OF THE PREFERRED EMBODIMENTS

The present invention provides bio-commemorative articles preferably associated with a famous individual, geographic location or historical event. The bio-commemorative articles are relatively inexpensive, and thus can be purchased by segments of the population that previously were precluded from obtaining an item associated with a famous individual, geographic location or historical event.

The bio-commemorative articles preferably contain amplified genetic material that either: is obtained from a famous individual; corresponds to a specific geographic location; or corresponds to a specific historical event. The genetic material is amplified by techniques well known to those of ordinary skill in the art, to thereby provide large quantities of amplified genetic material for use in the bio-commemorative articles.

In one preferred embodiment, the genetic material is DNA. The DNA may be nuclear DNA or mitochondrial DNA (mtDNA). Nuclear DNA is found in the nucleus of cells, and bodily fluids typically will be analyzed for nuclear DNA. When available bodily fluids from an individual provide an excellent source of nuclear DNA. Mitochondrial DNA is located in structures, called mitochondria, found in the outer layer of the cell. While the nucleus of the cell contains two copies of nuclear DNA, cells may contain hundreds of mitochondria, each of which may contain several copies of mtDNA. Thus, mtDNA has a greater copy number than nuclear DNA. This characteristic of mtDNA proves useful in situations where the amount of sample is very limited, e.g., in hair with no attached root. More recently, even nuclear DNA has been obtained root shafts in sufficient quantity for analysis. See, for example, Chang et al., Cancer Epidemiology, Biomarkers and Prevention, 11:925-929 (2002), the content of which is incorporated herein by reference. However, when the DNA is obtained from terminal hair shafts the DNA preferably is mtDNA.

Typical sources of nuclear DNA includes bodily fluids, whereas mtDNA often is extracted from hairs without tissue, bones, and teeth. For example, MtDNA has been extracted from degraded and old hair samples, including burnt specimens (Baker et al., J. Forensic Sci., 46:126-130 (2001), 100-year old Native American samples (Baker et al., PhD Thesis, University of Tennessee, Knoxville, 2001), and wool from a 9,400 Bighorn sheep Bonnichsen et al., J. ArcheoL Sci., 28:775-785 (2001). DNA may also be obtained from teeth with a high probability of recovering viable DNA, as tooth pulp encased in enamel is often the last part of a body to degrade. Even sweat or skin cells from an article of clothing worn by the individual may provide the source of the DNA.

The DNA, either nuclear or mitochondrial, is amplified and cloned using polymerase chain reaction (PCR). PCR amplification is how geneticists make infinitely small pieces of DNA reproduce themselves into much larger samples that can be easily worked within a laboratory. The PCR process is described in greater detail in U.S. Pat. No. 4,683,202 issued to Mullis, the content of which is incorporated herein by reference.

Samples are cleaned prior to amplification and cloning to remove contaminating materials surrounding or adhering to the sample. This step ensures that the sequence of the DNA obtained from the sample originates from the sample and not from exogenous human DNA.

The cleaning process for hair samples uses a detergent treatment in an ultrasonic water bath, which removes possible contaminating residues from the hair. The hair sample is then placed in an extraction solution and ground using a small mortar and pestle, resulting in a mixture that contains both the cellular material and the released DNA.

Bone and tooth samples also undergo a cleaning process. To clean a bone or tooth, an analyst sands the exterior to remove any extraneous material that may adhere to the surface. Then, the analyst removes a small sample, grinds it into a fine powder, and places the powdered bone and teeth in a solution to release the DNA from the cells.

To extract DNA, analysts expose the cellular mixture from the sample preparation step to a mixture of organic chemicals that separate the DNA from other biological material, such as proteins. The DNA sample is then purified to prepare it for the amplification process, which makes many copies of the target DNA.

In order to use PCR, one must already know the exact sequences which flank (lie on either side of) both ends of a given region of interest in DNA (may be a gene or any sequence). One need not know the DNA sequence in-between. The first step in PCR is the synthesis of known flanking sequences, called primers, of about 20 letters-long using a DNA synthesizer. The primers can be constructed in the lab, or purchased from commercial suppliers. Since the building-block sequences (nucleotide sequences) of many genes and the flanking regions of many genes are known, standard primers are available.

There are three basic steps in PCR. First, the target genetic material must be denatured-that is, the strands of its helix must be unwound and separated-by heating to 90-96° C. The second step is hybridization or annealing, in which the primers bind to their complementary bases on the now single-stranded DNA. The third step is DNA synthesis by a polymerase, an enzyme which can read the sequence of the opposing strand and extend the primer's sequence by adding individual bases together in the order in which they pair across from one another. One such enzyme used in PCR is called Taq polymerase. The result is two new helixes in place of the first, each composed of one of the original strands plus the newly assembled complementary strand.

To produce more DNA, the cycle is repeated, beginning with denaturing the DNA already made. The amount of DNA will double with each cycle. Each cycle takes only 1-3 minutes, so repeating the process for just 45 minutes can generate millions of copies of a specific DNA strand. Thus, PCR can do in a week work that used to take a year. Equipment for performing PCR is readily available, and the machines are now automated, in order to use the optimum temperature for each of the three steps.

Conventionally, the unmatched ability of the PCR technique to identify and copy the tiniest amounts of even old and damaged DNA has proved exceptionally valuable in the law, where it is used to provide enough DNA for sequencing so that evidence may be matched with an individual. It also has been used to establish evolutionary relationships among extinct species and present-day species. According to the present invention, PCR is used to generate large quantities of DNA for incorporation into bio-commemorative articles. In the present context, it is not necessary to sequence the DNA obtained following amplification. If other DNA from the individual or a close relative is available, the DNA may be sequenced in order to authenticate the samples. However, sequencing is not required, and authentication can be provided by providing documentation of chain of title for the source of the DNA. Most typically the bio-commemorative article containing DNA will be combined with documentation establishing chain of title, thereby authenticating the origin of the material as being the famous individual.

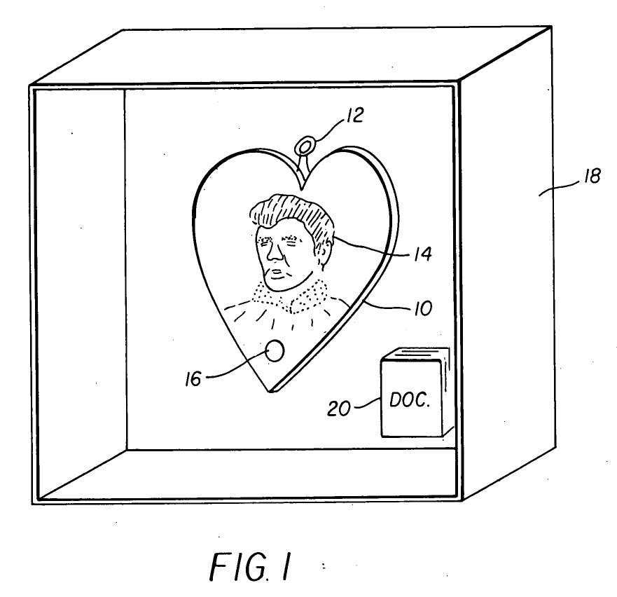

The DNA thus produced can be packaged in an article of manufacture, which may take any number of forms. For example, the article of manufacture may be an charm 10; designed to be worn by an individual as shown in FIG. 1. In the illustrated embodiment, the charm 10 is in the shape of a heart and includes an eye-ring 12 such that the charm 10 may be placed on a chain (not shown). The body of the charm 10 constitutes a support structure on which the amplified genetic material is incorporated. The charm 10 preferably includes an image 14 of the individual from which the genetic material is taken. Alternatively, the article of manufacture may be a vase, box, picture, painting or any other inanimate article in which the amplified genetic material is included, attached to or combined with in any form. The article of manufacture optionally may also have a name of the individual printed or embossed on its surface in addition to an image.

In the preferred embodiment, amplified the genetic material is incorporated into the material making up the charm 10, however, a chamber 16 or any other suitable mechanism may alternately be provided on the charm 10 to hold the genetic material in the form of a liquid, gel, solid or powder. Still further, the genetic material may be coated, printed or painted on the body of the charm 10. For purposes of this discussion, any process that overlays the amplified genetic material on the inanimate object such as painting, printing, silk-screening, etc., will be considered “coating” processes.

Since the volume of the actual amplified DNA is miniscule, it may be dissolved or suspended in a liquid or gel, mixed with a powder, or added to mixture that will solidify into a solid object that forms the bio-commemorative article. The material to which it is added may be colored or colorless, and transparent or opaque. Accordingly, the amplified genetic material may be incorporated directly into a support structure by molding or casting processes.

The bio-commemorative article may be packaged in a container 18 with authenticity documentation 20 establishing the authenticity of the amplified DNA with respect to its' original source. For example, the authenticity documentation 20 may include evidence of a chain of title for the material from which the original DNA sample was extracted from the moment the original sample was taken until the amplified DNA was incorporated with an inanimate object to form the commemorative article. Alternatively, the authenticity documentation 20 may include evidence of a comparison to DNA from a close relative of the individual.

It should be noted that the sample DNA does not necessarily have to be directly related to an individual. For example, DNA from plants or animals from a geographic location associated with the individual may be utilized. As just a few examples, DNA from flowers or plants growing on the gravesite of a famous individual, DNA from plants located at a famous religious site or DNA from flowers taken from a funeral wreath of an individual may be employed. In such cases, a bio-commemorative article containing the DNA would emotionally link the person purchasing the bio-commemorative article with a particular individual, a particular geographic location or a particular historical event.

In all cases, it would be preferable to obtain permission from an authenticating authority to allow direct samples of DNA to be extracted from a site of interest or an individual of interest. In the event that permission is not granted, however, the sample DNA could originate from a location adjacent to the site or from a relative related to the individual. In such cases, the portion of amplified DNA would preferably, although not necessarily, represent the same characteristics of the site or individual primarily of interest.

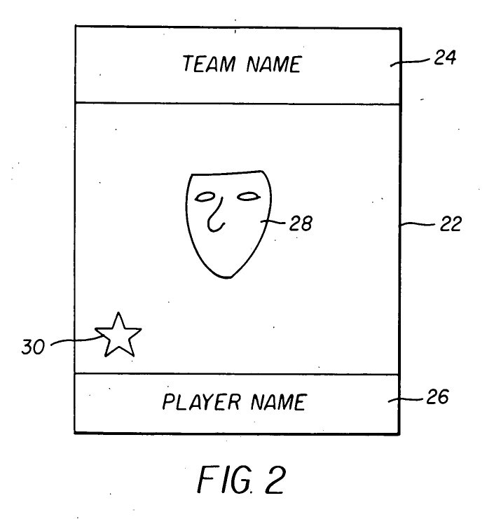

As stated above, the bio-commemorative article can take many forms. Since the DNA may be incorporated into liquids or gels, it is possible to make printed commemorative articles containing the DNA. For example, the DNA may be incorporated into ink that is used to print a picture, stamp, postcard, poster, book or other similar articles. As just one example, FIG. 2 illustrates a bio-commemorative baseball card 22 containing a header 24 in which the team name is displayed, a footer 26 in which a player's name is displayed, and a player's image 28 printed (for purposes of this application printing, coating and painting processes will be considered as being the same as state above) with ink containing amplified DNA of the player. In this example, the conventional card stock constitutes the support structure for the amplified genetic material contained in the ink. A seal 30 of authenticity is also preferably provided to authenticate that the card contains amplified DNA of the player depicted on the card.

Many modifications and variations may be made to the techniques and structures described and illustrated herein without departing from the spirit and scope of the invention. Accordingly, it should be understood that the bio-commemorative articles described herein are illustrative only and are not limiting upon the scope of the invention. For example, the method of amplification of the original genetic material is not limited to the use of PRC. Still further, the commemorative article need not be limited to solid support structures, but instead, also includes liquids such as perfume into which the genetic material is mixed. In such a case, the liquid carrier is considered a “support structure” to the genetic material for the purposes of this discussion.

What is claimed is:

1. A bio-commemorative article comprising:
a support structure; and
amplified genetic material from an organism incorporated with the support structure.

2. A bio-commemorative article as claimed in claim 1, wherein the amplified genetic material is amplified DNA.

3. A bio-commemorative article as claimed in claim 2, wherein the amplified DNA is polymerase chain reaction amplified DNA.

4. A bio-commemorative article as claimed in claim 1, wherein the amplified genetic material is incorporated into a material from which the support structure is manufactured.

5. A bio-commemorative article as claimed in claim 1, wherein the amplified genetic material is coated onto the support structure.

6. A bio-commemorative article as claimed in claim 1, wherein the support structure includes a holding chamber to hold the amplified genetic material.

7. A bio-commemorative article as claimed in claim 6, wherein the amplified genetic material is incorporated into one of a solid, a liquid or a gel.

8. A bio-commemorative article as claimed in claim 1, further comprising authenticity documentation.

9. A bio-commemorative article as claimed in claim 1, wherein the support structure includes a picture of an individual on its surface, wherein the amplified genetic material corresponds to the individual.

10. A bio-commemorative article as claimed in claim 1, wherein the support structure includes a name of an individual on its surface, wherein the amplified genetic material corresponds to the individual.

11. A bio-commemorative article as claimed in claim 1, wherein the amplified genetic material corresponds to a geographic location.

12. A bio-commemorative article as claimed in claim 1, wherein the amplified genetic material corresponds to an event.

13. A method of manufacturing a bio-commemorative article comprising:
obtaining a sample of genetic material of an organism;
amplifying the genetic material; and
incorporating the amplified genetic material with an inanimate object.

14. A method of manufacturing a bio-commemorative article as claimed in claim 13, wherein the genetic material is DNA and is amplified using a polymerase chain reaction process.

15. A method of manufacturing a bio-commemorative article as claimed in claim 13, wherein the amplified genetic material is coated on the inanimate object.

16. A method of manufacturing a bio-commemorative article as claimed in claim 13, wherein the amplified genetic material is included in a structure of the inanimate object.

17. A method of manufacturing a bio-commemorative article as claimed in claim 13, wherein the amplified genetic material is place in a chamber of the inanimate object.

18. A method of manufacturing a bio-commemorative article as claimed in claim 13, wherein the amplified genetic material is incorporated with one of a solid, a liquid, a gel or a powder.

19. A method of manufacturing a bio-commemorative article as claimed in claim 13, further comprising placing an image of an individual on the inanimate object, wherein the genetic material corresponds to the individual.

20. A method of manufacturing a bio-commemorative article as claimed in claim 13, further comprising placing a name of an individual on the inanimate object, wherein the genetic material corresponds to the individual.
